(19) United States
(12) Patent Application Publication
(10) Pub. No.: US 2011/0311630 A1
(43) Pub. Date: Dec. 22, 2011

(54) Novel embedment particles for inhalation

(75) Inventors: Michael Krueger, Ingelheim am Rhein (DE); Cordula Krueger, Ingelheim am Rhein (DE); Marc Egen, Ingelheim am Rhein (DE); Elke Jahr, Bingen (DE); Regis Cartier, Wiesbaden (DE); Anja Enderle, Muehltal (DE); Thierry Bouyssou, Warthausen (DE)

(73) Assignee: BOEHRINGER INGELHEIM INTERNATIONAL GMBH, Ingelheim am Rhein (DE)

(21) Appl. No.: 12/996,719

(22) PCT Filed: Jun. 8, 2009
(86) PCT No.: PCT/EP2009/057059

§ 371 Date: Jul. 26, 2011

(30) Foreign Application Priority Data
Jun. 9, 2008 (EP) 08104316.8

(51) Int. Cl. A61K 9/14

(52) U.S. Cl. 424/486

(57) ABSTRACT

The invention relates to the preparation of inhalable powders which exhibit a delayed release of active substance and processes for preparing them as well as medicaments that can be produced using these inhalable powders.

The invention relates to processes for preparing delayed-release medicaments and medicaments for administration by inhalation that may be produced by these processes. The invention relates in particular to poly-[lactide-co-glycolide]-based dry powder formulations which have a delayed release of active substance. The invention also relates to the use of these medicaments for the treatment of respiratory complaints, particularly for the treatment of COPD (chronic obstructive pulmonary disease) and asthma.

### BACKGROUND TO THE INVENTION

To achieve a reproducible and constant release of active substance it may be necessary to delay the release of active substance using special formulation techniques. In inhalative applications in particular in which the active substance after being administered by inhalation is present in finely divided form on the surface of the lungs for absorption, a rapid absorption of active substance is observed. This is reflected in a pharmacokinetic behaviour that corresponds to that observed after intravenous administration. From the field of the oral formulations it is known (R. H. Müller and G. E. Hildebrand: “Pharmazeutische Technologie: Moderne Arzneiformen”, Wissenschaftliche Verlagsgesellschaft mbH Stuttgart, 1997, ISBN 3-8047-1504-4), that the use of particular adjuvants may form an additional diffusion barrier to the process of the release of active substance or may interfere with the distribution of the active substance. This may occur as a result of
(i) the adjuvant forming a coating of particles of active substance or active substance microcompartments or
(ii) the adjuvant entering into interactions with the active substance, so that the latter is present in molecularly dispersed form in an adjuvant matrix.

Active substances are usually provided by oral administration. If this route is not suitable or not desirable on account of special properties of the active substance or particular demands made of the administration, various other possible methods of administering substances are known in the art.

In the form of powders for inhalation, inhalable powders packed for example into suitable capsules (inhalettes) are delivered to the lungs by means of powder inhalers. Other systems are also known in which the quantity of powder to be administered is pre-dosed (e.g. blisters), and multi-dose powder systems are also known. Alternatively, the medicaments may be administered by inhalation of suitable powdered inhalable aerosols which are suspended for example in HFA134a, HFA227 or mixtures thereof, as propellant gas.

During powder inhalation, the microparticles of the pure active substance are conventionally administered through the airways to the surface of the lungs, e.g. in the alveoli, by the inhalation process. These particles are deposited on the surface and are absorbed into the body directly after the dissolving process by active and passive transporting processes.

Inhalation systems are known in the literature in which the active substance is present in the form of solid particles, either as a micronised suspension in a suitable solvent system as carrier, or in the form of a dry powder.

Usually, powder inhalants, e.g. in the form of capsules for inhalation, are prepared on the basis of the general teaching, as described in DE-A-179 22 07.

A critical factor in multi-substance systems of this kind is the uniform distribution of the medicament in the powder mixture.

Another significant aspect with powder inhalants is that during the inhalative administration of the active substance only particles of a specific aerodynamic size reach the target organ, the lungs. The mean particle size of these lung-bound particles (inhalable fraction) is in the region of a few microns, typically between 0.1 and 10 μm, preferably below 6 μm. Particles of this kind are usually produced by micronisation (air jet milling).

It is known from the literature that particles in the region of a few microns may be prepared by spray-drying. Conventionally, formulations that can be handled industrially and which are sufficiently dispersible for medicinal administration (inhalation) are prepared from spray-dried particles of this kind using the process mentioned above (DE-A-179 22 07), [Y.-F. Maa, P.-A. Ngyuyen, J. D. Andya, N. Dasovich, T. D. Sweeny, S. J. Shire, C. C. Hsu, Pharmaceutical Research, 15, No. 5 (1998), 768-775; M. T. Vidgrén, P. A. Vidgrén, T. P. Paronen, Int. J. Pharmaceutics, 35 (1987), 139-144; R. W. Niven, F. D. Lott, A. Y. Ip, J. M. Cribbs, Pharmaceutical Research, 11, No. 8 (1994), 1101-1109].

The spray-drying of pure active substances for inhalation purposes (powder inhalation) is also described in the prior art [e.g.: EP 0 072 046 A1; WO 2000 000176 A1; U.S. Pat. No. 6,019,968; A. Chawla, K. M. G. Taylor, J. M. Newton, M. C. R. Johnson, Int. J. Pharm, 108 (3), (1994), 233-240].

Besides these examples, the pharmaceutical companies in particular make use of other manufacturing techniques based on spray-drying methods that describe special formulations for inhalable powders. The following may be mentioned as examples of these:

Powdered preparations consisting of co-spray-dried β-galactosidase with trehalose [J. Broadhead, S. K. Edmond Rouan, C. T. Rhodes, Pharm Acta Helvetiae, 70 (1995), 125-131], which may be mixed for example with other physiologically acceptable excipients; powdered preparations consisting of a spray micronisate which is obtained by co-spray-drying at least two active substances and one or more physiologically acceptable adjuvants [WO 01/13885]; powdered preparations consisting of spray-dried rhDNase, optionally co-spray-dried with salts, and prepared either directly or in the form of a mixture with a physiologically acceptable adjuvant e.g. lactose, mannitol or sodium chloride for inhalative administration [H. K. Chan, A. Clark, I Gonda, M. Mumenthaler, C. Hsu, Pharm Research, 14 (1997), 431-437]; spray-dried IGF1 preparations for inhalative administration [WO 9955362]; co-spray micronisates from active substances and physiologically acceptable adjuvants [WO 9952506] for inhalative administration; powdered preparations containing co-spray micronisates of SLPI protein in physiologically acceptable carrier materials [WO 9917800]; co-spray-dried interferon with a carrier material [WO 9531479]; co-spray micronisates comprising an active substance and cellulose derivatives [WO 9325198]; co-spray micronisates, consisting of RhDNase and a physiologically acceptable adjuvant, e.g. lactose, the initially amorphous adjuvant being converted into crystalline α-lactose monohydrate by subsequent recrystallisation [H.-K. Chan, I. Gonda, J. Pharm. Sci., 87 (5), (1998) 647-654].

### AIM OF THE INVENTION

Conventional manufacturing technologies for preparing embedding particles for administration by inhalation are based on the use of physiologically acceptable adjuvants. In the prior art the adjuvants that are used in powdered inhalants serve primarily to ensure that a uniform mixture and hence dilution of the active substance can be achieved using the adjuvants.

The aim of the invention is to enable a controlled release of the active substance to take place using the inhalable powder according to the invention. The invention therefore sets out to provide inhalable powders which have a time-delayed solution rate (delayed release) compared with particles of the pure active substance.

By delayed release is meant here that particles according to the invention have release characteristics such that particles display delayed dissolution characteristics in a Franz-type diffusion cell. As a consequence, a slower and at the same time long-lasting release of a pharmaceutical active substance from the inhalable powder according to the invention is observed, preferably from particles that have an aerodynamic size of less than 5 μm.

It is thus an aim of the invention to provide inhalable powders which have a delayed dissolution rate compared with the pure active substance particles as well as processes for preparing them. The invention sets out particularly to provide the above-mentioned inhalable powders for low molecular active substances, as well as for water-soluble active substances.

In another aspect the invention relates to the preparation of delayed-release inhalable powders which contain a biodegradable chemically modified polymer and processes for the preparation thereof.

Moreover the invention relates to the preparation of delayed-release inhalable powders which consist exclusively of a low molecular active substance and a biodegradable polymer and processes for the preparation thereof.

The invention also sets out to provide medicaments which contain inhalable powders according to the invention.

### DETAILED DESCRIPTION OF THE INVENTION

During the preparation of an inhalable powder for pulmonary (or nasal) inhalation the active substance (or a physiologically acceptable salt thereof) is incorporated in physically stable form as a solid in a solid matrix of an adjuvant.

By a corresponding choice of adjuvants, using the formulation technique according to the invention the active substance may be incorporated in the solid matrix such that it has a delayed release. By this is meant, according to the invention, that the solution characteristics of the inhalable particles in a release medium are delayed by comparison with inhalable particles of the pure active substance, determined in a Franz diffusion cell.

The inhalable fraction represents the amount of inhalable active substance particles (particles<5 μm) that can be determined on the basis of the Pharm. Eur. 2.9.18 (European Pharmacopoeia, 6th edition 2008, Apparatus D—Andersen Cascade Impactor) or USP30-NF25<601>. The inhalable fraction is also referred to within the scope of the present invention as the FPD (Fine Particle Dose).

Surprisingly it has been found that the inhalable particles of the inhalable powders according to the invention solve the problems stated above if the active substance or several active substances is or are incorporated in an adjuvant matrix and the adjuvant is selected from among the block copolymers which contain at least one hydrophilic and one hydrophobic block. In particular, the problem is solved if the active substance or several active substances is or are incorporated in an adjuvant matrix and the adjuvant is selected from among the PEG-modified (poly-[lactide-co-glycolide]) based polymers (hereinafter referred to as PEG-modified PLGA). These substances may be obtained for example under the name Resomer® (Boehringer Ingelheim Pharma GmbH & Co. KG, Germany). Substances from this category are particularly suitable if they
have a PEG content of 1-15%, preferably 1-10%, preferably 1-5%
a molecular mass of between 37.5 and 600 kDa
a diblock structure or triblock structure
a glycolide content of 0-50%
and a D-lactide content of 0-25%
   and are in the form of a block copolymer, preferably with a diblock structure (A-B) or a triblock structure (A-B-A). These contain at least one water-soluble block (block B) and at least one non-water-soluble block (block A). By a diblock structure or triblock structure is meant that the polymer is made up of different units which are repeated regularly at a molecular level. PEG (polyethyleneglycol) is used in particular as the water-soluble block. A polyester compound is used in particular as the non-water-soluble block. For example, the category of polymers poly-(lactide-co-glycolide) is used as the polyester block.

Of particular importance are the PEG-[lactide-co-glycolides] listed in Table 1 as embedding materials for inhalable powders according to the invention.

TABLE 1
Suitable embedding materials for the preparation of inhalable powders
according to the invention (manufacturer: Boehringer Ingelheim);
M_{w} = molecular weight in [kD] (calculated theoretically according to the
manufacturing process on the basis of the quantities of monomer used),
T_{g} = glass transition temperature in [° C.].

| Name | Structure | (weight or molar) Composition | M_{w} | T_{g} |
|---|---|---|---|---|
| RGP d 5055 | diblock | 5 kDa-PEG-[D,L-lactide/ | 100 | 39 |
| | | glycolide] 5%/47.5%/47.5% | | |
| LRP d 7055 | diblock | 5 kDa-PEG-[D-lactide/L-lactide] | 100 | 41 |
| | | 5%/15.2%/79.8% | | |
| LGP d 8555 | diblock | 5 kDa-PEG-[L-lactide/glycolide] | 100 | 44 |
| | | 5%/77.9%/17.1% | | |
| LRP t 7046 | triblock | [D-lactide/L-lactide]-6 kDa- | 150 | 49 |
| | | PEG-[D-lactide/L-lactide] | | |
| | | 7.68%/40.32%/4%/7.68%/ | | |
| | | 40.32% | | |
| LRP t 7016 | triblock | [D-lactide/L-lactide]-6 kDa- | 600 | n.d. |
| | | PEG-[D-lactide/L-lactide] | | |
| | | 6.93%/42.57%/1%/6.93%/ | | |
| | | 42.57% | | |
| LGP t 8546 | triblock | [L-lactide/glycolide]-6 kDa- | 150 | 45 |
| | | PEG-[L-lactide/glycolide] | | |
| | | 39.36%/8.64%/4%/39.36%/ | | |
| | | 8.64% | | |
| LGP t 8516 | triblock | [L-lactide/glycolide]-6 kDa- | 600 | n.d. |
| | | PEG-[L-lactide/glycolide] | | |
| | | 41.09%/8.4%/4%/41.09%/8.4% | | |
| LP t 52 | triblock | L-lactide-2 kDa-PEG-L-lactide | 40 | n.d. |
| | | 47.5%/5%/47.5% | | |
| LGP t 5046 | triblock | [L-lactide/glycolide]-6 kDa- | 150 | 43 |
| | | PEG-[L-lactide/glycolide] | | |
| | | 23.52%/24.48%/4%/23.52%/ | | |
| | | 24.48% | | |

According to the invention the inhalable powders (based on the inhalable fraction (particles with an aerodynamic particle size of less than 5 μm)) are characterised in that at most 90% of the active substance has gone into solution after 10 hours, preferably at most 90% of the active substance has dissolved after 12 hours.

In another embodiment, inhalable powders according to the invention (based on the inhalable fraction (particles with an aerodynamic particle size of less than 5 μm)) are characterised in that 40% to 80% of the active substance, preferably 50% to 80% of the active substance, more preferably 60% to 80% of the active substance and still more preferably 70% to 80% of the active substance goes into solution within less than 200 minutes.

Also, in another embodiment, inhalable powders, based on their inhalable fraction, are characterised in that 40% to 80% of the active substance, preferably 50% to 80% of the active substance, more preferably 60% to 80% of the active substance and still more preferably 70% to 80% of the active substance goes into solution within less than 120 minutes.

Also, in another embodiment, inhalable powders, based on their inhalable fraction, are characterised in that 40% to 80% of the active substance, preferably 50% to 80% of the active substance, more preferably 60% to 80% of the active substance and still more preferably 70% to 80% of the active substance goes into solution within less than 60 minutes.

The solution characteristics of the inhalable fraction of the inhalable powder according to the invention serve as a measurement of the delayed release of the active substance.

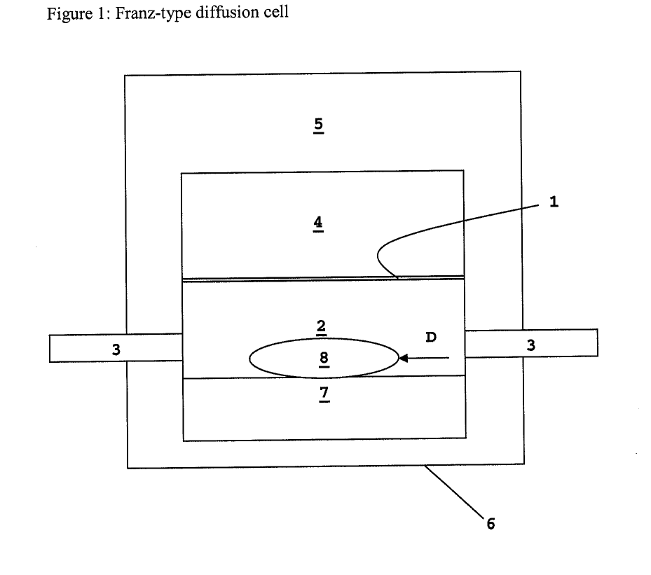

These solution characteristics may be determined using a Franz diffusion cell (cf. FIG. 1). A lower compartment is filled with a release medium which can be freely selected, and the membrane (in this case a filter membrane) is placed on the surface of the medium, ensuring that no air is still trapped between the release medium and the membrane. The upper part of the cell closes off the system and forms an air compartment.

In this embodiment the lower compartment is connected to a pump by tubes that carry the medium to a device for measurement data acquisition, for example a UV detector or a fluorescence detector. An active substance can be quantitatively detected using detectors of this kind.

Finally, the release medium is mixed with a stirrer system such as a magnetic stirrer in order to distribute an active substance taken up in the release medium more evenly inside the chamber.

[numerical data in the next section refer to FIG. 1] The inhalable fraction of the inhalable powders according to the invention is deposited in finely divided form on a filter membrane 1 in a Franz diffusion cell. Underneath the membrane 6 is disposed a first compartment 2 for receiving a liquid release medium free from air bubbles, which reacts continuously, as indicated by the connectors 3 and the throughflow arrow D, and a device for measurement data acquisition, such as a UV or fluorescence detector. Above the membrane 1 an air chamber is formed as the second compartment 4, and the entire diffusion cell 5 (Franz cell) is surrounded by thermal insulation 6 and can be temperature controlled in the desired manner by means of a hotplate 7. The release medium is mixed by means of a magnetic stirrer 8.

Preferably the inhalable fraction of the inhalable powders according to the invention may be deposited on a cellulose membrane. The depositing of the inhalable fraction may be preferably carried out by placing this filter on the filter plate of the Andersen Cascade Impactor. Delivery is then carried out in accordance with Pharm. Eur. 2.9.18 (European Pharmacopoeia, 6th edition 2008, Apparatus D—Andersen Cascade Impactor), while only the deposition plates that are not used for the deposition of particles from 0 to 5 μm in size are placed in the cascade impactor, so that all the particles smaller than 5 μm are deposited on the filter.

The invention further relates to processes by which the problems according to the invention are solved. The invention comprises corresponding manufacturing methods for producing inhalable powders according to the invention. Such powders may be used both directly as powdered inhalants (multi-dose systems, pre-metered multi-dose systems and single dose systems) and also as components which are mixed with other (e.g. coarse-grained) adjuvants.

In order to produce such particles, the manufacturing method may be controlled so as to obtain the particles in a suitable particle size, usually between 0.1 and 10 μm, and so that the particles have surface qualities that make them easy to swirl and disperse.

In all, a formulation based on this manufacturing method enables the active substance or a physiologically acceptable salt thereof to be administered to the patient by inhalation in a therapeutically useful dose as a delayed-release medicament.

The particles of the inhalable powders according to the invention which are prepared by the process according to the invention are characterised by high physical stability. They are particularly suitable if a high fine content is delivered when they are used as powdered inhalants, determined technically, e.g. by measurement with a cascade impactor. Typically the proportion of the particles produced by this method that are smaller than 5 μm (aerodynamically) is greater than 15%; in some cases, fine contents of more than 30%, or more than 50%, are obtained.

Powders thus produced are characterised by a particle size, e.g. measured by laser diffraction, by a mean particle size X₅₀ in the range from 1 μm to 10 μm, preferably from 1 μm to 6 μm. By the mean particle size X₅₀ in the sense used here is meant the 50% value from the volume distribution, measured with a laser diffractometer by the dry dispersion method.

The manufacturing method for the microparticles or inhalable powders according to the invention is characterised in that a solution or emulsion of the active substance or a physiologically acceptable salt thereof is suitably dissolved or processed to form an emulsion with an adjuvant selected from among the PEG-modified (poly-[lactide-co-glycolide]) based polymers (PEG-modified PLGA), which is then sprayed and dried in a spraying tower. The particles/the powder may be obtained by a suitable deposition process (e.g. cyclone or fine particle filter). The microparticles thus prepared are characterised by special values in terms of their particle size.

For active substances that have a water solubility of more than 0.01 g per 100 mL, it has proved appropriate, when preparing microparticles in the form of embedding particles of the inhalable powders according to the invention, to use a process that comprises the following steps:
(a) preparing a (W/O) emulsion, wherein the active substance(s) are dissolved in water and preferably using dichloromethane as the organic phase, in which a triblock copolymer (PLGA-PEG-PLGA) is dissolved.
(b) spraying the resulting emulsion in the usual way, to obtain a spray mist with a droplet size having a characteristic value X₅₀ of between 7 μm and 25 μm,
(c) drying the spray mist thus obtained using a drying gas, while applying the following parameters:
   (i) an entry temperature for the drying gas of from 30° C. to 350° C., preferably from 40° C. to 250° C. and particularly preferably from 45° C. to 150° C., and
   (ii) an exit temperature of the drying gas of from 30° C. to 120° C., and
(d) separating the dried solid particles from the drying gas current in the usual way.

For active substances which have a solubility of more than 0.01 g per 100 mL in an organic solvent which is completely water-miscible, it has proved suitable, when preparing microparticles in the form of embedding particles of the inhalable powders according to the invention, to use a process that comprises the following steps:
(a) preparing a solution of organic solvent which has unlimited miscibility with water, by dissolving the active substance or substances with a diblock copolymer (PEG-PLGA), preferably with a glycolide content of 0%, in the solvent,
(b) spraying the resulting solution in the usual way, to obtain a spray mist with a droplet size having a characteristic value X₅₀ of between 7 μm and 25 μm,
(c) drying the spray mist thus obtained using a drying gas, while applying the following parameters:
   an entry temperature for the drying gas of from 30° C. to 350° C., preferably from 40° C. to 250° C. and particularly preferably from 145° C. to 150° C., and
   an exit temperature of the drying gas of from 30° C. to 120° C., and
(d) separating the dried solid particles from the drying gas current in the usual way.

For active substances which have a solubility of more than 0.01 g per 100 mL in an organic solvent, preferably dichloromethane, it has proved suitable, when preparing microparticles in the form of embedding particles of the inhalable powders according to the invention, to use a process that comprises the following steps:
(a) preparing a solution in which the active substance or substances and a PEG-PLGA block copolymer are dissolved in the solvent,
(b) spraying the resulting solution in the usual way, to obtain a spray mist with a droplet size having a characteristic value X₅₀ of between 7 μm and 25 μm,
(c) drying the spray mist thus obtained using a drying gas, while applying the following parameters:
   (i) an entry temperature for the drying gas of from 30° C. to 350° C., preferably from 40° C. to 250° C. and particularly preferably from 145° C. to 150° C., and
   (ii) an exit temperature of the drying gas of from 30° C. to 120° C., and
(d) separating the dried solid particles from the drying gas current in the usual way.

The particle sizes were determined within the scope of the present invention by laser diffraction (Fraunhofer diffraction). By the mean particle size X₅₀ in the sense used here is meant the 50% value from the volume distribution. More detailed information on this can be found in the experimental descriptions of the invention.

According to the invention the inhalable powders thus obtained may be used for preparing a medicament. They are preferably used to prepare a medicament for treating respiratory complaints, particularly for treating COPD and/or asthma. The invention also relates to the use of the inhaler powders thus obtained for preparing a medicament for use by inhalation, particularly for preparing a medicament for inhalation which allows a delayed release of the active substance.

The chemical compounds listed hereinafter (active substances) may be used on their own or in combination as the medicament-relevant component of the inhalable powders according to the invention.

In the compounds mentioned below, W is a pharmacologically active substance and is selected (for example) from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors. Moreover, double or triple combinations of W may be combined and used in the device according to the invention. Combinations of W might be, for example:
W denotes a betamimetic, combined with an anticholinergic, corticosteroid, PDE4-inhibitor, EGFR-inhibitor or LTD4-antagonist,
W denotes an anticholinergic, combined with a betamimetic, corticosteroid, PDE4-inhibitor, EGFR-inhibitor or LTD4-antagonist,
W denotes a corticosteroid, combined with a PDE4-inhibitor, EGFR-inhibitor or LTD4-antagonist
W denotes a PDE4-inhibitor, combined with an EGFR-inhibitor or LTD4-antagonist
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5,6-diethyl-indan-2-ylamino)-1-hydroxy-ethyl]-8-hydroxy-1H-quinolin-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl)-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol
5-hydroxy-8-(1-hydroxy-2-isopropylaminobutyl)-2H-1,4-benzoxazin-3-(4H)-one
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2,4,6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl)-1,1-dimethyl-ethylamino]-ethyl}-4H-benzo[1,4]oxazin-3-one
6-hydroxy-8-{1-hydroxy-2-[2-(4-isopropyl-phenyl)-1.1dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3,4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
2-hydroxy-5-(1-hydroxy-2-{2-[4-(2-hydroxy-2-phenyl-ethylamino)-phenyl]-ethylamino}-ethyl)-benzaldehyde
N-[2-hydroxy-5-(1-hydroxy-2-{2-[4-(2-hydroxy-2-phenyl-ethylamino)-phenyl]-ethylamino}-ethyl)-phenyl]-formamide
8-hydroxy-5-(1-hydroxy-2-{2-[4-(6-methoxy-biphenyl-3-ylamino)-phenyl]-ethylamino}-ethyl)-1H-quinolin-2-one
8-hydroxy-5-[1-hydroxy-2-(6-phenethylamino-hexylamino)-ethyl]-1H-quinolin-2-one
5-[2-(2-{4-[4-(2-amino-2-methyl-propoxy)-phenylamino]-phenyl}-ethylamino)-1-hydroxy-ethyl]-8-hydroxy-1H-quinolin-2-one
[3-(4-{6-[2-hydroxy-2-(4-hydroxy-3-hydroxymethyl-phenyl)-ethylamino]-hexyloxy}-butyl)-5-methyl-phenyl]-urea
4-(2-{6-[2-(2,6-dichloro-benzyloxy)-ethoxy]-hexylamino}-1-hydroxy-ethyl)-2-hydroxymethyl-phenol
3-(4-{6-[2-hydroxy-2-(4-hydroxy-3-hydroxymethyl-phenyl)-ethylamino]-hexyloxy}-butyl)-benzylsulphonamide
3-(3-{7-[2-hydroxy-2-(4-hydroxy-3-hydroxymethyl-phenyl)-ethylamino]-heptyloxy}-propyl)-benzylsulphonamide
4-(2-{6-[4-(3-cyclopentanesulphonyl-phenyl)-butoxy]-hexylamino}-1-hydroxy-ethyl)-2-hydroxymethyl-phenol
N-adamantan-2-yl-2-(3-{2-[2-hydroxy-2-(4-hydroxy-3-hydroxymethyl-phenyl)-ethylamino]-propyl}-phenyl)-acetamide
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other preferred anticholinergics are selected from among the salts of formula AC-1

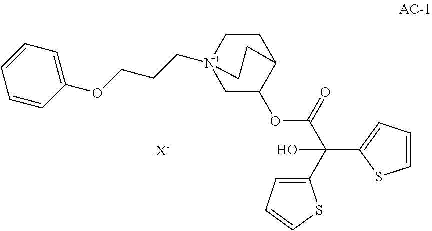

wherein X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-ene

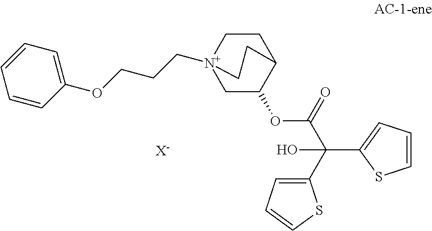

wherein X⁻ may have the above-mentioned meanings. Other preferred anticholinergics are selected from the salts of formula AC-2

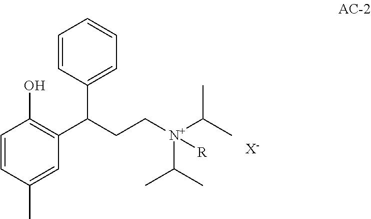

wherein R denotes either methyl or ethyl and wherein X⁻ may have the above-mentioned meanings. In an alternative embodiment the compound of formula AC-2 may also be present in the form of the free base AC-2-base.

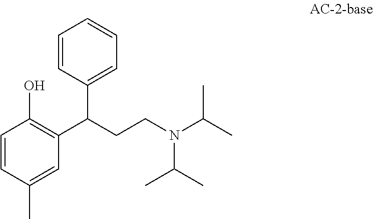

Other specified compounds are:
tropenol 2,2-diphenylpropionate methobromide
scopine 2,2-diphenylpropionate methobromide
scopine 2-fluoro-2,2-diphenylacetate methobromide
tropenol 2-fluoro-2,2-diphenylacetate methobromide
tropenol 3,3′,4,4′-tetrafluorobenzilate methobromide
scopine 3,3′,4,4′-tetrafluorobenzilate methobromide
tropenol 4,4′-difluorobenzilate methobromide
scopine 4,4′-difluorobenzilate methobromide
tropenol 3,3′-difluorobenzilate methobromide
scopine 3,3′-difluorobenzilate methobromide
tropenol 9-hydroxy-fluorene-9-carboxylate methobromide
tropenol 9-fluoro-fluorene-9-carboxylate methobromide
scopine 9-hydroxy-fluorene-9-carboxylate methobromide
scopine 9-fluoro-fluorene-9-carboxylate methobromide
tropenol 9-methyl-fluorene-9-carboxylate methobromide
scopine 9-methyl-fluorene-9-carboxylate methobromide
cyclopropyltropine benzilate methobromide;
cyclopropyltropine 2,2-diphenylpropionate methobromide
cyclopropyltropine 9-hydroxy-xanthene-9-carboxylate methobromide
cyclopropyltropine 9-methyl-fluorene-9-carboxylate methobromide
cyclopropyltropine 9-methyl-xanthene-9-carboxylate methobromide
cyclopropyltropine 9-hydroxy-fluorene-9-carboxylate methobromide
cyclopropyltropine methyl 4,4′-difluorobenzilate methobromide
tropenol 9-hydroxy-xanthene-9-carboxylate methobromide
scopine 9-hydroxy-xanthene-9-carboxylate methobromide
tropenol 9-methyl-xanthene-9-carboxylate methobromide
scopine 9-methyl-xanthene-9-carboxylate methobromide
tropenol 9-ethyl-xanthene-9-carboxylate methobromide
tropenol 9-difluoromethyl-xanthene-9-carboxylate methobromide
scopine 9-hydroxymethyl-xanthene-9-carboxylate methobromide

The above-mentioned compounds may also be used as salts within the scope of the present invention, wherein instead of the methobromide the salts metho-X are used, wherein X may have the meanings given hereinbefore for X⁻.

As corticosteroids it is preferable to use compounds selected from among beclomethasone, betamethasone, budesonide, butixocort, ciclesonide, deflazacort, dexamethasone, etiprednol, flunisolide, fluticasone, loteprednol, mometasone, prednisolone, prednisone, rofleponide, triamcinolone, RPR-106541, NS-126, ST-26 and
(S)-fluoromethyl 6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl)6,9-difluoro-11-hydroxy-16-methyl-3-oxo-17-propionyloxy-androsta-1,4-diene-17-carbothionate,
cyanomethyl 6α,9α-difluoro-11β-hydroxy-16α-methyl-3-oxo-17α-(2,2,3,3-tertamethylcyclopropylcarbonyl)oxy-androsta-1,4-diene-17β-carboxylate
   optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, CI-1018, CDC-801, CDC-3052, D-22888, YM-58997, Z-15370 and
—N-(3,5-dichloro-1-oxo-pyridin-4-yl)-4-difluoromethoxy-3-cyclopropylmethoxybenzamide
(−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N-2-cyano-S-m ethyl-isothioureido]benzyl)-2-pyrrolidone
cis[4-cyano-4-(3-cyclopentyloxy-4-methoxyphenyl)cyclohexane-1-carboxylic acid]-2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3,4-c]-1,2,4-triazolo[4,3-a]pyridine
9-cyclopentyl-5,6-dihydro-7-ethyl-3-(tert-butyl)-9H-pyrazolo[3,4-c]-1,2,4-triazolo[4,3-a]pyridine
   optionally in the form of the racemates, enantiomers or diastereomers thereof and optionally in the form of the pharmacologically acceptable acid addition salts thereof, the solvates and/or hydrates thereof. According to the invention the acid addition salts of the PDE4 inhibitors are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid
[2-[[2-(4-tert-butyl-2-thiazolyl)-5-benzofuranyl]oxymethyl]phenyl]acetic acid
   optionally in the form of the racemates, enantiomers or diastereomers thereof and optionally in the form of the pharmacologically acceptable acid addition salts, solvates and/or hydrates thereof. According to the invention these acid addition salts are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palm itates, pivalates or furoates.

EGFR-inhibitors which may be used are preferably compounds selected from among cetuximab, trastuzumab, ABX-EGF, Mab ICR-62 and
4-[(3-chloro-4-fluorophenyl)amino]-6-{[4-(morpholin-4-yl)-1-oxo-2-buten-1-yl]amino}-7-cyclopropylmethoxy-quinazoline
4-[(3-chloro-4-fluorophenyl)amino]-6-{[4-(N,N-diethylamino)-1-oxo-2-buten-1-yl]-amino}-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopropylmethoxy-quinazoline
4-[(R)-(1-phenyl-ethyl)amino]-6-{[4-(morpholin-4-yl)-1-oxo-2-buten-1-yl]amino}-7-cyclopentyloxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-chloro-4-fluoro-phenyl)amino]-6-[2-((S)-6-methyl-2-oxo-morpholin-4-yl)-ethoxy]-7-methoxy-quinazoline
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopentyloxy-quinazoline
4-[(R)-(1-phenyl-ethyl)amino]-6-{[4-(N,N-to-(2-methoxy-ethyl)-amino)-1-oxo-2-buten-1-yl]amino}-7-cyclopropylmethoxy-quinazoline
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline
4-[(3-chloro-4-fluorophenyl)amino]-6-{[4-(N-cyclopropyl-N-methyl-amino)-1-oxo-2-buten-1-yl]amino}-7-cyclopentyloxy-quinazoline
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
4-[(3-ethynyl-phenyl)amino]-6.7-to-(2-methoxy-ethoxy)-quinazoline
4-[(3-chloro-4-fluorophenyl)amino]-7-[3-(morpholin-4-yl)-propyloxy]-6-[vinyl-carbonyl)amino]-quinazoline
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline
4-{[3-chloro-4-(3-fluoro-benzyloxy)-phenyl]amino}-6-(5-{[(2-methanesulphonyl-ethyl)amino]methyl}-furan-2-yl)quinazoline
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline
4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline
4-[(3-chloro-4-fluorophenyl)amino]-6-({4-[N,N-to-(2-methoxy-ethyl)-amino]-1-oxo-2-buten-1-yl}amino)-7-[tetrahydrofuran-2-yl)methoxy]-quinazoline
4-[(3-ethynyl-phenyl)amino]-6-{[4-(5.5-dimethyl-2-oxo-morpholin-4-yl)-1-oxo-2-buten-1-yl]amino}-quinazoline
4-[(3-chloro-4-fluoro-phenyl)amino]-6-[2-(2,2-dimethyl-6-oxo-morpholin-4-yl)-ethoxy]-7-methoxy-quinazoline
4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline
4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline
4-[(3-chloro-4-fluoro-phenyl)amino]-6-{2-[4-(2-oxo-morpholin-4-yl)-piperidin-1-yl]-ethoxy}-7-methoxy-quinazoline
4-[(3-chloro-4-fluoro-phenyl)amino]-6-[1-(tert.-butyloxycarbonyl)-piperidin-4-yloxy]-7-methoxy-quinazoline
4-[(3-chloro-4-fluoro-phenyl)amino]-6-(trans-4-amino-cyclohexan-1-yloxy)-7-methoxy-quinazoline
4-[(3-chloro-4-fluoro-phenyl)amino]-6-(trans-4-methanesulphonylamino-cyclohexan-1-yloxy)-7-methoxy-quinazoline
4-[(3-chloro-4-fluoro-phenyl)amino]-6-(tetrahydropyran-3-yloxy)-7-methoxy-quinazoline
4-[(3-chloro-4-fluoro-phenyl)amino]-6-(1-methyl-piperidin-4-yloxy)-7-methoxy-quinazoline
4-[(3-chloro-4-fluoro-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[methoxymethyl)carbonyl]-piperidin-4-yloxy}-7-methoxy-quinazoline
4-[(3-chloro-4-fluoro-phenyl)amino]-6-(piperidin-3-yloxy)-7-methoxy-quinazoline
4-[(3-chloro-4-fluoro-phenyl)amino]-6-[1-(2-acetylamino-ethyl)-piperidin-4-yloxy]-7-methoxy-quinazoline
4-[(3-chloro-4-fluoro-phenyl)amino]-6-(tetrahydropyran-4-yloxy)-7-ethoxy-quinazoline
4-[(3-chloro-4-fluoro-phenyl)amino]-6-((S)-tetrahydrofuran-3-yloxy)-7-hydroxy-quinazoline
4-[(3-chloro-4-fluoro-phenyl)amino]-6-(tetrahydropyran-4-yloxy)-7-(2-methoxy-ethoxy)-quinazoline
4-[(3-chloro-4-fluoro-phenyl)amino]-6-{trans-4-[(dimethylamino)sulphonylamino]-cyclohexan-1-yloxy}-7-methoxy-quinazoline
4-[(3-chloro-4-fluoro-phenyl)amino]-6-{trans-4-[(morpholin-4-yl)carbonylamino]-cyclohexan-1-yloxy}-7-methoxy-quinazoline
4-[(3-chloro-4-fluoro-phenyl)amino]-6-{trans-4-[(morpholin-4-yl)sulphonylamino]-cyclohexan-1-yloxy}-7-methoxy-quinazoline
4-[(3-chloro-4-fluoro-phenyl)amino]-6-(tetrahydropyran-4-yloxy)-7-(2-acetylamino-ethoxy)-quinazoline
4-[(3-chloro-4-fluoro-phenyl)amino]-6-(tetrahydropyran-4-yloxy)-7-(2-methanesulphonylamino-ethoxy)-quinazoline
4-[(3-chloro-4-fluoro-phenyl)amino]-6-{1-[(piperidin-1-yl)carbonyl]-piperidin-4-yloxy}-7-methoxy-quinazoline
4-[(3-chloro-4-fluoro-phenyl)amino]-6-(1-aminocarbonylmethyl-piperidin-4-yloxy)-7-methoxy-quinazoline
4-[(3-chloro-4-fluoro-phenyl)amino]-6-(cis-4-{N-[tetrahydropyran-4-yl)carbonyl]-N-methyl-amino}-cyclohexan-1-yloxy)-7-methoxy-quinazoline
4-[(3-chloro-4-fluoro-phenyl)amino]-6-(cis-4-{N-[morpholin-4-yl)carbonyl]-N-methyl-amino}-cyclohexan-1-yloxy)-7-methoxy-quinazoline
4-[(3-chloro-4-fluoro-phenyl)amino]-6-(cis-4-{N-[morpholin-4-yl)sulphonyl]-N-methyl-amino}-cyclohexan-1-yloxy)-7-methoxy-quinazoline
4-[(3-chloro-4-fluoro-phenyl)amino]-6-(trans-4-ethanesulphonylamino-cyclohexan-1-yloxy)-7-methoxy-quinazoline
4-[(3-chloro-4-fluoro-phenyl)amino]-6-(1-methanesulphonyl-piperidin-4-yloxy)-7-ethoxy-quinazoline
4-[(3-chloro-4-fluoro-phenyl)amino]-6-(1-methanesulphonyl-piperidin-4-yloxy)-7-(2-methoxy-ethoxy)-quinazoline
4-[(3-chloro-4-fluoro-phenyl)amino]-6-[1-(2-methoxy-acetyl)-piperidin-4-yloxy]-7-(2-methoxy-ethoxy)-quinazoline
4-[(3-chloro-4-fluoro-phenyl)amino]-6-(cis-4-acetylamino-cyclohexan-1-yloxy)-7-methoxy-quinazoline
4-[(3-ethynyl-phenyl)amino]-6-[1-(tert.-butyloxycarbonyl)-piperidin-4-yloxy]-7-methoxy-quinazoline
4-[(3-ethynyl-phenyl)amino]-6-(tetrahydropyran-4-yloxy]-7-methoxy-quinazoline
4-[(3-chloro-4-fluoro-phenyl)amino]-6-(cis-4-{N-[piperidin-1-yl)carbonyl]-N-methyl-amino}-cyclohexan-1-yloxy)-7-methoxy-quinazoline
4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-chloro-4-fluoro-phenyl)amino]-6-{cis-4-[(morpholin-4-yl)carbonylamino]-cyclohexan-1-yloxy}-7-methoxy-quinazoline
4-[(3-chloro-4-fluoro-phenyl)amino]-6-{1-[2-(2-oxopyrrolidin-1-yl)ethyl]-piperidin-4-yloxy}-7-methoxy-quinazoline
4-[(3-chloro-4-fluoro-phenyl)amino]-6-{1-[morpholin-4-yl)carbonyl]-piperidin-4-yloxy}-7-(2-methoxy-ethoxy)-quinazoline
4-[(3-ethynyl-phenyl)amino]-6-(1-acetyl-piperidin-4-yloxy)-7-methoxy-quinazoline
4-[(3-ethynyl-phenyl)amino]-6-(1-methyl-piperidin-4-yloxy)-7-methoxy-quinazoline
4-[(3-ethynyl-phenyl)amino]-6-(1-methanesulphonyl-piperidin-4-yloxy)-7-methoxy-quinazoline
4-[(3-chloro-4-fluoro-phenyl)amino]-6-(1-methyl-piperidin-4-yloxy)-7(2-methoxy-ethoxy)-quinazoline
4-[(3-chloro-4-fluoro-phenyl)amino]-6-(1-isopropyloxycarbonyl-piperidin-4-yloxy)-7-methoxy-quinazoline
4-[(3-chloro-4-fluoro-phenyl)amino]-6-(cis-4-methylamino-cyclohexan-1-yloxy)-7-methoxy-quinazoline
4-[(3-chloro-4-fluoro-phenyl)amino]-6-{cis-4-[N-(2-methoxy-acetyl)-N-methyl-amino]-cyclohexan-1-yloxy}-7-methoxy-quinazoline
4-[(3-ethynyl-phenyl)amino]-6-(piperidin-4-yloxy)-7-methoxy-quinazoline
4-[(3-ethynyl-phenyl)amino]-6-[1-(2-methoxy-acetyl)-piperidin-4-yloxy]-7-methoxy-quinazoline
4-[(3-ethynyl-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(cis-2,6-dimethyl-morpholin-4-yl)carbonyl]-piperidin-4-yloxy}-7-methoxy-quinazoline
4-[(3-chloro-4-fluoro-phenyl)amino]-6-{1-[(2-methyl-morpholin-4-yl)carbonyl]-piperidin-4-yloxy}-7-methoxy-quinazoline
4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline
4-[(3-chloro-4-fluoro-phenyl)amino]-6-{1-[(N-methyl-N-2-methoxyethyl-amino)carbonyl]-piperidin-4-yloxy}-7-methoxy-quinazoline
4-[(3-chloro-4-fluoro-phenyl)amino]-6-(1-ethyl-piperidin-4-yloxy)-7-methoxy-quinazoline
4-[(3-chloro-4-fluoro-phenyl)amino]-6-{1-[(2-methoxyethyl)carbonyl]-piperidin-4-yloxy}-7-methoxy-quinazoline
4-[(3-chloro-4-fluoro-phenyl)amino]-6-{1-[(3-methoxypropyl-amino)-carbonyl]-piperidin-4-yloxy}-7-methoxy-quinazoline
4-[(3-chloro-4-fluoro-phenyl)amino]-6-[cis-4-(N-methanesulphonyl-N-methyl-amino)-cyclohexan-1-yloxy]-7-methoxy-quinazoline
4-[(3-chloro-4-fluoro-phenyl)amino]-6-[cis-4-(N-acetyl-N-methyl-amino)-cyclohexan-1-yloxy]-7-methoxy-quinazoline
4-[(3-chloro-4-fluoro-phenyl)amino]-6-(trans-4-methylamino-cyclohexan-1-yloxy)-7-methoxy-quinazoline
4-[(3-chloro-4-fluoro-phenyl)amino]-6-(trans-4-(N-methanesulphonyl-N-methyl-amino)-cyclohexan-1-yloxy)-7-methoxy-quinazoline
4-[(3-chloro-4-fluoro-phenyl)amino]-6-(trans-4-dimethylamino-cyclohexan-1-yloxy)-7-methoxy-quinazoline
4-[(3-chloro-4-fluoro-phenyl)amino]-6-(trans-4-{N-[morpholin-4-yl)carbonyl]-N-methyl-amino}-cyclohexan-1-yloxy)-7-methoxy-quinazoline
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline
4-[(3-chloro-4-fluoro-phenyl)amino]-6-(1-methanesulphonyl-piperidin-4-yloxy)-7-methoxy-quinazoline
4-[(3-chloro-4-fluoro-phenyhamino]-6-(1-cyano-piperidin-4-yloxy)-7-methoxy-quinazoline
   optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptine, cabergoline, alpha-dihydroergocryptine, lisuride, pergolide, pramipexole, roxindole, ropinirole, talipexole, terguride and viozan, optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorphenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The pharmaceutically effective substances, formulations or mixtures of substances used may be any inhalable compounds, including also for example inhalable macromolecules, as disclosed in EP 1 003 478. Preferably, substances, formulations or mixtures of substances for treating respiratory complaints which are administered by inhalation are used.

In addition, the compound may come from the group of ergot alkaloid derivatives, the triptans, the CGRP-inhibitors, the phosphodiesterase-V inhibitors, optionally in the form of the racemates, enantiomers or diastereomers thereof, optionally in the form of the pharmacologically acceptable acid addition salts, the solvates and/or hydrates thereof.

Examples of ergot alkaloid derivatives are dihydroergotamine and ergotamine.

### EXPERIMENTAL SECTION

### (1) Methods of Measurement

a) Determining Particle Size by Laser Diffraction (Average Particle Size x₅₀):

### Measuring Device and Settings:

The apparatus are operated in accordance with the manufacturers operating instructions.
Measuring device: Laser diffraction spectrometer (HELOS), Sympatec (particle sizes measured by Fraunhofer diffraction)
Dispersing unit: RODOS dry disperser with suction funnel, Sympatec
Sample quantity: 200 mg±150 mg
Product feed: Vibri vibrating channel, made by Sympatec
Frequency of vibrating channel: rising to 100
Duration of sample feed: 15 to 25 sec. (in the case of 200 mg)
Focal length: 100 mm (measuring range: 0.9-175 μm)
Measuring time/waiting time: approx. 15 s (in the case of 200 mg)
Cycle time: 20 ms
Start/stop at: 1% on channel 28
Dispersing gas: compressed air
Pressure: 3 bar
Vacuum: maximum
Evaluation method: HRLD

### Sample Preparation/Product Feed:

About 200 mg of the test substance are weighed onto a piece of card.

Using another piece of card all the larger lumps are broken up. The powder is then sprinkled finely over the front half of the vibrating channel (starting about 1 cm from the front edge). After the start of the measurement the frequency of the vibrating channel is varied so that the sample is fed as continuously as possible. However, the quantity of product must not be too great, so as to ensure that adequate dispersion is achieved.

### b) Determining the Droplet Size by Laser Diffraction

Measuring method: To determine the droplet size the spray cone of the nozzle is analysed directly in the laser measuring zone with respect to the droplet size distribution. By the median value X₅₀ is meant the droplet size below which 50% of the quantity of droplets fall. H₂O is used as the test solution to determine suitable nozzle parameters.
Measuring device: Laser diffraction spectrometer (HELOS), Sympatec
Software: WINDOX Version 4
Dispersing unit: RODOS/dispersing pressure: 3 bar
Focal length: 100 mm [measuring range: 0.9 . . . 175 μm]
Evaluation method: Mie (V 4)

### b) Determining the Emulsion Droplet Size by Photon Correlation Spectroscopy (Zetasizer, Malvern)

Measuring device: Zetasizer, Malvern, type Zetasizer Nano ZS
Software: Dispersion Technology Software Version 4.10 (Malvern)

### Measuring Conditions/Measuring Parameters Method:

Measuring processes according to the manufacturer's instructions. The measuring device calculates the hydrodynamic diameter (Dh) of a suspension and gives the size distribution. The results of measurement listed below correspond to the respective main peaks of the size distributions determined.

### (2) Examples

a) Dry Powder Formulations which Contain a Water-Soluble Active Substance

These are produced using a spray dryer made by Büchi, of the B-290 mini-spray dryer type. The dry powder formulations listed in Table 2 were obtained by preparing w/o (water in DCM) emulsions which were spray-dried. The emulsions were prepared using an ultrasound apparatus (made by Sonics & Materilas Inc., Vibra Cell type, fitted with a 3 mm tip). To prepare the emulsion the tip is dipped 0.5-2 cm into the solution and the ultrasound apparatus is operated at 30%.

TABLE 2
Dry powder formulations (ID = identification code)

| | | active substance/ | Solvent(s)/ | x₅₀ |
|---|---|---|---|---|
| ID | polymer(s) | load in [%] | Additives | [μm] |
| MP7/SR-1 | LRP t 7046 | salbutamol sulphate/20% | H₂O/DCM | 3.5 |
| SR-2 | LGP t 8546 | salbutamol sulphate/20% | H₂O/DCM | 3.4 |
| SR-2.7 | LGP t 8546 | salbutamol sulphate/20% | H₂O/DCM | 2.2 |
| SR-6.1 | RGP d 5055 | salbutamol sulphate/20% | H₂O/DCM | 2.6 |
| SR-11 | LP t 52 | salbutamol sulphate/20% | H₂O/DCM | 2.5 |
| SR-12 | LGP d 8555 | salbutamol sulphate/20% | H₂O/DCM | 3.4 |
| SR-13 | LRP d 7055 | salbutamol sulphate/20% | H₂O/DCM | 3.3 |
| SR-14 | LGP t 5046 | salbutamol sulphate/20% | H₂O/DCM | 2.3 |
| SBR-2.0 | LGP t 8546 | salbutamol base/20% | acetone | 1.7 |
| SBR-3.0 | RGP d 5055 | salbutamol base/20% | acetone | 2.3 |
| SRME-1.0 | LGP t 8546 | salbutamol sulphate/20% | H₂O/DCM/ | 0.95 |
| | | | ethanol | |

b) Dry Powder Formulations which Contain a Non-Water-Soluble Active Substance

The dry powder formulations listed in Table 3 are obtained by spray-drying solutions of the polymer and of the active substance budesonide.

TABLE 3
Dry powder formulations with budesonide

| ID | polymer | solvent | x₅₀ [μm] |
|---|---|---|---|
| BR-1.2 | LGP t 8546 | DCM | 2.00 |
| BR2 | LRP t 7046 | DCM | 2.27 |
| BR3 | LP t 52 | DCM | 1.39 |

### Release Characteristics of Inhalable Powders According to the Invention

The inhalable fraction of the inhalable powders according to the invention was investigated in a dissolution model (Franz-type diffusion cell) with regard to the controlled release of salbutamol or budesonide. (In order to consider the particle fraction that would be deposited in the lungs in human application from a HandiHaler®, particles>5 μm were differentiated by using stages 0 and 1 of the cascade impactor).

The inhalable powders discussed in Examples 1 to 5 that follow, distinguishable by their respective identification codes, were obtained by the spray-drying method. The respective process parameters are listed in Table 4.

TABLE 4
Process parameters for the inhalable powders discussed in Examples 1 to 5.

| | identification code ID | | | | | | | |
|---|---|---|---|---|---|---|---|---|
| Setting parameters | SR 2.8 | SR 11 | SR 14 | SR 1.3 | SR 12.0 | SR 13.0 | SR 6.1 | SBR 2.0 |
| Entry temperature [° C.] | 94-100 | 75-100 | 65-75 | 79-95 | 52-67 | 68-78 | 69-75 | 72-77 |
| Exit temperature [° C.] | 47-53 | 35-39 | 45 | 47-53 | 35-39 | 45 | 35-38 | 40-42 |
| Delivery pump | 45 | 45 | 30 | 45 | 45 | 40 | 45 | 60 |
| performance [%] | | | | | | | | |
| N₂ spray flow [Nl/min] | 29-30 | 29-31 | 29-30 | 29-30 | 29-30 | 29-30 | 29-30 | 29-30 |
| Nozzle gas [bar] | 4.5 | 4.5 | 4.5 | 4.5 | 4.5 | 4.5 | 4.5 | 4 |
| Aspirator [%] | 100 | 100 | 100 | 100 | 100 | 100 | 100 | 100 |
| Differential pressure | 60 | 60 | 60 | 60 | 60 | 60 | 60 | 60 |
| [mbar] | | | | | | | | |

| | identification code ID | | | | |
|---|---|---|---|---|---|
| Setting parameters | BR 1.2 | SRME 1.0 | SBR 3.0 | BR 2.0 | BR 3.0 |
| Entry temperature [° C.] | 85-89 | 59-65 | 60-66 | 80-116 | 47-72 |
| Exit temperature [° C.] | 46-49 | 35-37 | 35-38 | 43-47 | 35-39 |
| Delivery pump performance [%] | 100 | 60 | 60 | 100 | 80 |
| N₂ spray flow [Nl/min] | 15-16 | 26 | 29-30 | 32-33 | 32-32 |
| Nozzle gas [bar] | 2 | 3.5 | 4.5 | 4.5 | 4.5 |
| Aspirator [%] | 100 | 100 | 100 | 100 | 100 |
| Differential pressure [mbar] | 60 | 60 | 60 | 60 | 60 |

### Example 1

Embedding particles (identification code SR 2.8; SR 11; SR 14; SR 1.3) were prepared by spray-drying from the active substance salbutamol together with different triblock copolymers.

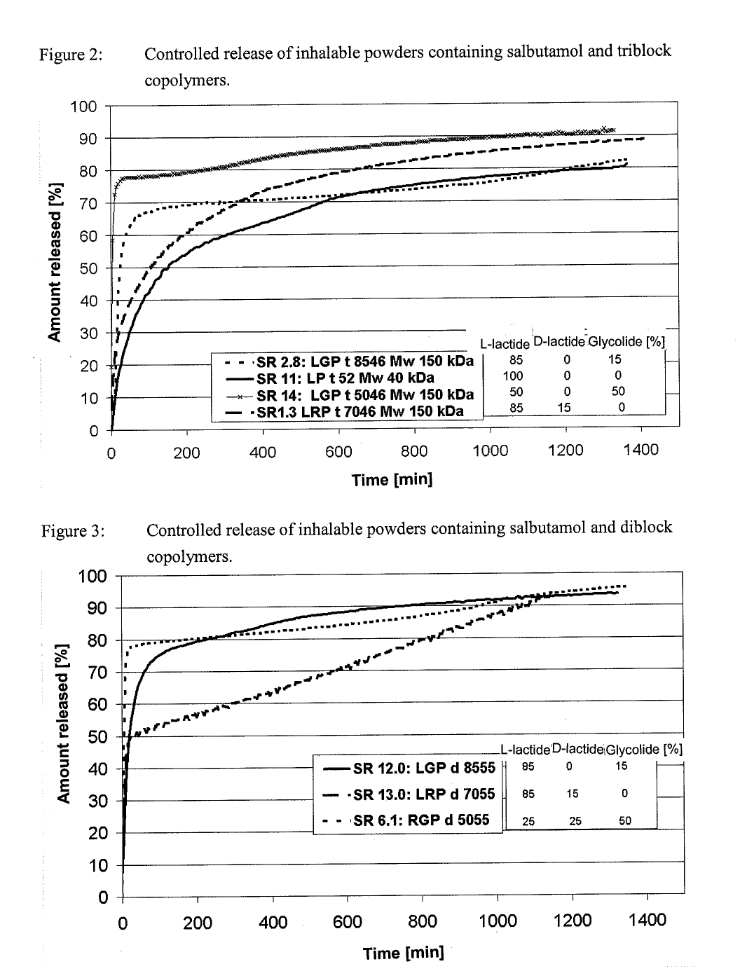

FIG. 2 shows the release characteristics (37° C., release medium PBS buffer (phosphate-buffered solution)) of the active substance in the inhalable fraction of the inhalable powders according to the invention. LGP t 8546; LP t 52; LGP t 5046 and LRP t 7046 were used as triblock copolymers. All the particles exhibited a delayed release over 24 hours.

### Example 2

Embedding particles (identification code SR 12.0; SR 13.0; SR 6.1) were prepared by spray-drying from the active substance salbutamol together with different diblock copolymers.

FIG. 3 shows the release characteristics (37° C., release medium PBS buffer (phosphate-buffered solution)) of the active substance in the inhalable fraction of the inhalable powders according to the invention. LGP d 8555; LRP d 7055 and RGP d 5055 were used as diblock copolymers. All the particles exhibited a delayed release over 24 hours.

### Example 3

Embedding particles were prepared by spray-drying from the active substance salbutamol together with the triblock copolymer. In the sample with the identification code SBR 2.0 the spray-drying was carried out from a homogeneous solution of the active substance and of the polymer (embedding material: triblock copolymer LGP t 8546) in acetone. The sample with the identification code SR 2.8, on the other hand, was prepared by producing a W/O emulsion, with the active substance dissolved in the aqueous phase and using dichloromethane (containing triblock copolymer LGP t 8546 dissolved therein as embedding material) as the organic phase.

The sample with the identification code SRME was prepared by adding further ethanol to the W/O emulsion (water/dichloromethane) until the emulsion clarified. Measurements using dynamic light scattering with an apparatus made by Malvern, of the Zetasizer nano ZS type, showed that for microemulsions characteristic double peaks were observed at 12 nm and 300 nm, which remained stable for at least 45 minutes.

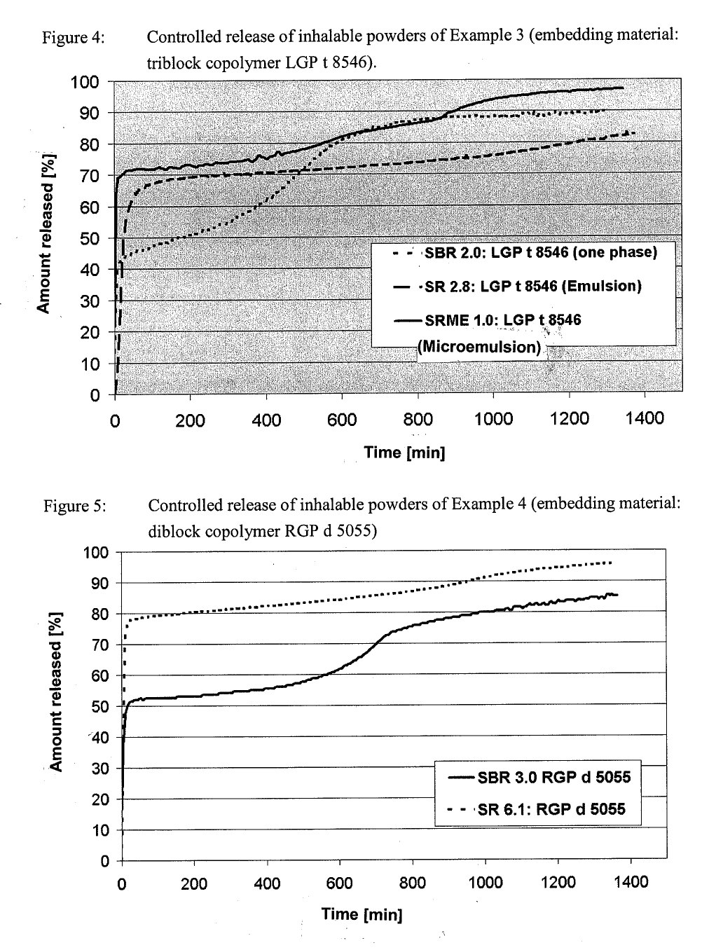

FIG. 4 shows the release characteristics (37° C., release medium PBS buffer (phosphate-buffered solution)) of the active substance in the inhalable fraction of the inhalable powders according to the invention.

### Example 4

Other inhalable embedding particles (identification code SBR 3.0; SR 6.1) were prepared by spray-drying from the active substance salbutamol together with the diblock copolymer RGP d 5055.

FIG. 5 shows the release characteristics (37° C., release medium PBS buffer (phosphate-buffered solution)) of the active substance in the inhalable fraction of the inhalable powders according to the invention.

### Example 5

Other inhalable embedding particles (identification code BR 3.0, BR 4.0; BR 2.0; BR 1.2) were prepared by spray-drying from the active substance budesonide together with the triblock copolymers LP t 52; LRP t 7046 and LGP t 8546.

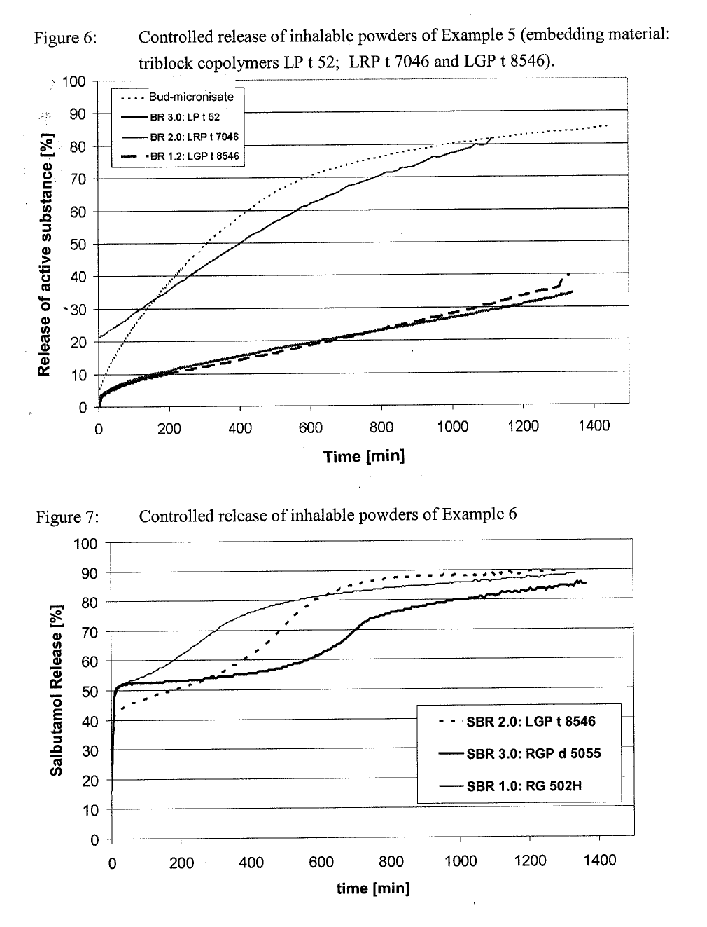

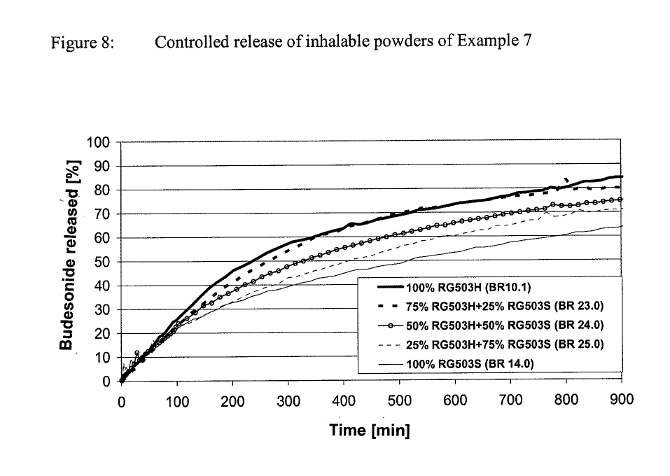

FIG. 6 shows the release characteristics (37° C., release medium PBS buffer (phosphate-buffered solution)) of the active substance in the inhalable fraction of the inhalable powders according to the invention.

1. Inhalable powder for administration by pulmonary or nasal inhalation, characterised in that it contains microparticles in which one or more active substances are incorporated in an adjuvant matrix and the adjuvant is selected from among the PEG-modified PLGA.

2. Inhalable powder according to claim 1, characterised in that the adjuvant selected from among the PEG-modified PLGA contains a PEG fraction of 1-15%, a molecular mass of 37.5-150 kDa, corresponds to a diblock (PEG-PLGA) or triblock structure (PLGA-PEG-PLGA), has a glycolide content of 0-50%, and a D-lactide content of 0-25%.

3. Inhalable powder according to claim 1, characterised in that it is prepared by a spray-drying process.

4. Process for preparing microparticles in the form of embedding particles according to claim 1, containing one or more active substances that have a water-solubility of more than 0.01 g per 100 mL and an adjuvant selected from among the PEG-modified PLGA, the process comprising the steps of
(a) preparing a (W/O) emulsion, wherein the active substance(s) is or are dissolved in water and dichloromethane is preferably used as the organic phase in which a triblock copolymer (PLGA-PEG-PLGA) is dissolved,
(b) spraying the solution thus obtained in the conventional manner, so as to obtain a spray mist with a droplet size having a characteristic value x₅₀ of between 7 μm and 25 μm,
(c) drying the spray mist thus obtained using a drying gas, while applying the following parameters:
(i) an entry temperature of the drying gas of 30° C. to 350° C., preferably 40° C. to 250° C. and particularly preferably 45° C. to 150° C. and
(ii) an exit temperature of the drying gas of 30° C. to 120° C. and
(d) separating the dried solid particles from the drying gas current in conventional manner.

5. Process for preparing microparticles in the form of embedding particles according to claim 1, containing one or more active substances, by dissolving the active substance(s) in an organic solvent which has unlimited miscibility with water, the active substance(s) having a solubility in this solvent of more than 0.01 g per 100 mL, and dissolving an adjuvant selected from among the PEG-modified PLGA therein, comprising the steps of
(a) preparing a solution, by dissolving the active substance(s) and a diblock copolymer (PEG-PLGA), preferably with a glycolide content of 0%, in an organic solvent,
(b) spraying the resulting solution in conventional manner so as to obtain a spray mist with a droplet size having a characteristic value x₅₀ of between 7 μm and 25 μm,
(c) drying the spray mist thus obtained using a drying gas, while applying the following parameters:
(i) an entry temperature of the drying gas of 30° C. to 350° C., preferably 40° C. to 250° C. and particularly preferably 45° C. to 150° C. and
(ii) an exit temperature of the drying gas of 30° C. to 120° C. and
(d) separating the dried solid particles from the drying gas current in conventional manner.

6. Process for preparing microparticles in the form of embedding particles according to claim 1, containing one or more active substances which have a solubility in an organic solvent, preferably dichloromethane, of more than 0.01 g per 100 mL, and containing an adjuvant that is selected from among the PEG-modified PLGA, comprising the steps of
(a) preparing a solution containing the active substance(s) and a PEG-PLGA block copolymer,
(b) spraying the resulting solution in conventional manner so as to obtain a spray mist with a droplet size having a characteristic value x₅₀ of between 7 μm and 25 μm,
(c) drying the spray mist thus obtained using a drying gas, while applying the following parameters:
(i) an entry temperature of the drying gas of 30° C. to 350° C., preferably 40° C. to 250° C. and particularly preferably 45° C. to 150° C. and
(ii) an exit temperature of the drying gas of 30° C. to 120° C. and
(d) separating the dried solid particles from the drying gas current in conventional manner.

7. Inhalable powder which may be obtained by one of the processes according to claim 4.

8. Medicament, characterised in that it contains an inhalable powder according to claim 1.
